(19) United States
(12) Patent Application Publication
(10) Pub. No.: US 2024/0312636 A1
(43) Pub. Date: Sep. 19, 2024

(54) RISK STRATIFICATION INTEGRATING MHEALTH AND AI

(71) Applicant: WEST VIRGINIA UNIVERSITY BOARD OF GOVERNORS ON BEHALF OF WEST VIRGINIA UNIVERSITY, West Virginia, WV (US)

(72) Inventors: Partho SENGUPTA, Morgantown, WV (US); Naveena YANAMALA, Morgantown, WV (US)

(21) Appl. No.: 18/571,143

(22) PCT Filed: Jun. 16, 2022
(86) PCT No.: PCT/US2022/033734

§ 371 Date: Dec. 15, 2023

Related U.S. Application Data
(60) Provisional application No. 63/211,829, filed on Jun. 17, 2021.

(51) Int. Cl. G16H 50/30; A61B 8/08; A61B 8/14; G16H 30/40

(52) U.S. Cl. CPC G16H 50/30; A61B 8/0883; A61B 8/14; G16H 30/40

(57) ABSTRACT

Various examples are provided related to mHealth based risk stratification. In one example, a system includes a handheld echocardiography device that can generate ultrasound (US) images of a patient and processing circuitry comprising a processor and memory. The processing circuitry can receive the US images from the handheld echocardiography device; generate enhanced echo images from the US images using a generative adversarial network (GAN) model; and determine a major adverse cardiac event (MACE) risk for the patient based upon the enhanced echo images. In another example, a method includes receiving US images of a patient obtained with a handheld echocardiography device; generating enhanced echo images from the US images using a generative adversarial network (GAN) model; and determining a major adverse cardiac event (MACE) risk for the patient based upon the enhanced echo images.

### CROSS REFERENCE TO RELATED APPLICATIONS

This application claims priority to, and the benefit of, co-pending U.S. provisional application entitled “Risk Stratification Integrating mHealth and AI” having Ser. No. 63/211,829, filed Jun. 17, 2021, which is hereby incorporated by reference in its entirety.

### BACKGROUND

Appropriate and adequate phenotyping is essential to understand the natural history of disease and to plan and execute effective interventional measures. Many of the currently well-identified cardiovascular conditions are syndromic in disposition. Myocardial infarctions (MI) are among the leading causes of morbidity and mortality in the United States and are associated with extremely high burden (>$11 billion US$ annually) in hospitalization costs alone. Even though the incidence and the complications of acute myocardial infarction (AMI) have been declining in the United States, this condition remains a high health priority. There exists a wide variation in hospital practices and outcomes of AMI patients. Most importantly, the mosaic of AMI presentation is too complex to be homogenized into simplified clinical algorithms. Several interesting questions remain unanswered. For example, it is a common practice in the United States that AMI patients are discharged with a request for a follow-up visit after 1 week. It is unknown why all patients—irrespective of their predicted risk of adverse outcomes—should come for follow-up within one week. It is conceivable that such a non-personalized approach to follow-up would lead to high demands on a healthcare delivery system that is starved for time and will also consequently increase the healthcare costs.

### SUMMARY

Aspects of the present disclosure are related to mHealth based risk stratification. In one aspect, among others, a system comprises a handheld echocardiography device configured to generate ultrasound (US) images of a patient; and processing circuitry comprising a processor and memory, the processing circuitry configured to: receive the US images from the handheld echocardiography device; generate enhanced echo images from the US images using a generative adversarial network (GAN) model; and determine a major adverse cardiac event (MACE) risk for the patient based upon the enhanced echo images. In one or more aspects, the GAN model can comprise a sparse skip connection U-Net model. The sparse skip connection U-Net model combines an encoder-decoder model and a U-Net model.

In various aspects, the enhanced echo images can comprise an apical four-chamber (A4C) view, an apical two chamber (A2C) view, a parasternal long-axis (PLAX) view and a parasternal short-axis (PSAX) view. The MACE risk determination can comprise extracting features from the enhanced echo images; analyzing phenotypes based at least in part upon the extracted features; and predicting risk of the MACE using a machine learning model. The extracted features can include morphometric/texture-based and deep-learning based latent features. The phenotype analysis can comprise patient similarity analysis using topological data analysis (TDA) or other unsupervised approaches. The predicted risk can be based upon the patient similarity analysis and clinical information associated with the patient. Extracting features of the enhanced echo images can comprise identifying end-systolic (ES) and end-diastolic (ED) frames from the enhanced echo images and selecting regions of interest (ROIs) from the identified ED/ES frames; and performing texture-based analysis (radiomics) and speckle tracking for phenotyping heterogeneous presentation. The ES and ED frames can be identified using non-negative matrix factorization.

In another aspect, a method comprises receiving ultrasound (US) images of a patient obtained with a handheld echocardiography device; generating enhanced echo images from the US images using a generative adversarial network (GAN) model; and determining a major adverse cardiac event (MACE) risk for the patient based upon the enhanced echo images. The US images can be received from the handheld echocardiography device. In one or more aspects, the GAN model can comprise a sparse skip connection U-Net model. The sparse skip connection U-Net model combines an encoder-decoder model and a U-Net model.

In various aspects, the enhanced echo images can comprise an apical four-chamber (A4C) view, an apical two chamber (A2C) view, a parasternal long-axis (PLAX) view and a parasternal short-axis (PSAX) view. The MACE risk determination can comprise extracting features from the enhanced echo images; analyzing phenotypes based at least in part upon the extracted features; and predicting risk of the MACE using a machine learning model. The phenotype analysis can comprise patient similarity analysis using topological data analysis (TDA). The predicted risk can be based upon the patient similarity analysis and clinical information associated with the patient. Extracting features of the enhanced echo images can comprise identifying end-systolic (ES) and end-diastolic (ED) frames from the enhanced echo images and selecting regions of interest (ROIs) from the identified ED/ES frames; and performing texture-based analysis (radiomics) and speckle tracking for phenotyping heterogeneous presentation. The ES and ED frames can be identified using non-negative matrix factorization. The features extracted from corresponding ROIs of the identified ED/ES frames can comprise left ventricular (LV) geometry.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of texture patterns of ultrasound images and myofiber and matrix architecture, in accordance with various embodiments of the present disclosure.

FIG. 2 illustrates an example of a machine-learning (ML) pipeline, in accordance with various embodiments of the present disclosure.

FIGS. 3A and 3B illustrate an example of unsupervised patient clustering, in accordance with various embodiments of the present disclosure.

FIG. 4 illustrates an example of Kaplan-Meier curve analyses of a patient similarity network, in accordance with various embodiments of the present disclosure.

FIG. 5 illustrates an example of a speckle tracking echocardiography (STE), in accordance with various embodiments of the present disclosure.

FIG. 6 illustrates an example of information extraction from echocardiographic images using a radiomics guided deep neural network pipeline, in accordance with various embodiments of the present disclosure.

FIG. 7 illustrates an example of enhancement of POCUS images, in accordance with various embodiments of the present disclosure.

FIGS. 8A and 8B illustrate an example of underuse implications of echocardiography, in accordance with various embodiments of the present disclosure.

FIG. 9 illustrates an example of patient differentiation (without ST-elevation changes) using a POCUS-derived model, in accordance with various embodiments of the present disclosure.

FIG. 10 illustrates an example of hierarchical clustering of the echo-derived radiomic features, in accordance with various embodiments of the present disclosure.

FIG. 11 illustrates an example of a system that can be used for pocket US enhancement and risk stratification, in accordance with various embodiments of the present disclosure.

### DETAILED DESCRIPTION

Disclosed herein are various examples related to mHealth based risk stratification. Despite the concerns over a potential overuse of echocardiography, studies have shown that echocardiographic evaluation is underused in admitted AMI patients. It has been shown that patients who do not undergo echocardiographic evaluation during hospitalization are at an increased risk of adverse outcomes. Handheld echocardiography (e.g., pocket ultrasound) in unison with other electronic resources can bridge the “echocardiography gap” and can help to accurately risk stratify the AMI patients. Reference will now be made in detail to the description of the embodiments as illustrated in the drawings, wherein like reference numbers indicate like parts throughout the several views.

The more severe forms of acute myocardial infarction (AMI) like ST-segment elevation myocardial infarction (STEMI) are known to be associated with more adverse outcomes, longer hospital stays and higher hospitalization costs than the less severe forms (e.g., unstable angina). These observations from recent studies urge a quick reappraisal of the phenotyping practices—both from the perspective of accuracy and of standardization. Another area of importance with regard to AMI is that not all patients hospitalized for AMI undergo a cardiac imaging evaluation even when they have had a diagnostic cardiac catheterization done. There has been some concern over the potential overuse of imaging techniques like echocardiography in hospitalized AMI patients, but the reality is that almost 25%of the hospitalized AMI patients do not get an echocardiographic evaluation throughout their hospital stay.

Furthermore, the follow-up protocols for discharged AMI patients vary, entailing a first visit often encouraged within 7 days after discharge. This recommendation may not be complied with by several patients. Moreover, this recommendation is homogeneous across the phenotypes of AMI such that even low-risk patients need to make this follow-up visit that is clinically non-informative and infrastructurally demanding. Rationalization of such protocols by ensuring echocardiographically phenotyped AMI patients can thus substantially improve cost and resource burden for AMI hospitalizations. Together, these issues related to phenotypic variability, homogenized treatment and follow-up protocols, varied echocardiography protocols and inter individual variation in follow-up outcomes beckon a need for novel and effective alternatives.

Three facts can help rationalize the study of mHealth-based, deep-learning-driven models of AMI risk stratification. First, the convenience and portability of the mHealth devices (e.g., a pocket USG machine) in conducting a quick, bedside evaluation of patient makes a very attractive alternative to standard echocardiography. The value of handheld echocardiography devices in identification and classification of valvular heart diseases, heart structures, and left ventricular functional parameters is detailed in “Pocket-Sized Echocardiography Devices: One Stop Shop Service?” by Seraphim et al. (J Cardiovasc Ultrasound, 2016. 24(1): p. 1-6), which is hereby incorporated by reference in its entirety. Comparison of handheld echocardiography with standard echocardiography has shown that there exists a moderate-to-almost perfect correlation between the two modalities with respect to ejection fraction measurement, valve regurgitation identification, left ventricular function and regional wall motion abnormality.

Second, the pocket USG machine is unlikely to possess the full functionality with the same image quality as that obtained through standard echocardiography. For example, generative adversarial network (GAN) models can be employed to perform ultrasound image reconstruction to improve the imaging quality of portable ultrasound images. Improving the imaging quality with miniaturized imaging devices can be unified with machine learning techniques. Third, the use of electronic health records and electrocardiograms as prognosticators of AMI has been demonstrated. Finally, the addition of electrocardiographic data can further augment the precision phenotyping by adding complementary imaging related diagnostic and prognostic features. For example, the application of AI with signal-processed electrocardiography (ECG) can enhance predicting myocardial relaxation abnormalities.

Existing standard echocardiography measurements can be used with novel texture-based analysis (radiomics) and speckle tracking for phenotyping heterogeneous presentation in AMI and develop unsupervised and supervised models using machine learning methods for AMI risk prediction. This can set a benchmark model for prognostication of hospitalized AMI patients. Development of mHealth based risk stratification (focusing, e.g., on pocket-sized echocardiograph and combining these with clinical & ECG inputs) can be used as an alternate strategy for the phenotypic prediction. Novel deep-learning approaches can be utilized to enhance the image quality of pocket-sized echocardiograph with features extracted as concatenable inputs that are comparable in performance to the developed model. This can ensure that the mHealth-based alternatives are useful in appropriately triaging patients similar to the standard echocardiography-based model. The mHealth-integrated strategy may be compared with standard echocardiography strategy in AMI patients to predict clinically meaningful endpoints (e.g., primary endpoints like MACE, death; and secondary endpoints like readmissions and hospitalization costs).

Handheld echocardiography is a point-of-care alternative to standard echocardiography. This bedside evaluation, which can take <10 minutes, can be carried out at the time of discharge is less expensive as compared to the standard echocardiography. The benefits of handheld echocardiography can be combined with the power and accuracy of deep learning to provide risk-stratification strategies that are accurate, acceptable, and clinically meaningful. Echocardiographic features of LV geometry and function including speckle tracking strain and morphometric/texture-based features can be retrospectively extracted to develop AI-based echocardiography models that identify high-risk AMI features and predict adverse AMI outcomes. Prediction models can be developed using novel mHealth resources (e.g., pocket USG-based images and electronic medical record-based hospitalization data). Image reconstruction methods can be used to improve the image quality of handheld ultrasound equipment in terms of spatial resolution, contrast, and noise reduction.

After improving the quality of the point of care ultrasound (POCUS) images, features can be extracted and used with developed mHealth based applications. A standard echocardiography-based model and a mHealth based model can be developed that predicts poor clinical outcomes like MACE, hospital and short-term mortality and the frequency and risk of readmissions and the associated hospital costs. Adding an mHealth-based evaluation at the time discharge offers an alternative to the standard-of-care recommendation of performing pre-discharge or early (within 7 days) follow-up echocardiographic evaluation in every AMI patient.

AMI phenotyping challenges: Current Universal Definition of Myocardial Infraction (MI) uses pathophysiology-based classification that differentiates injury from ischemia and atherothrombotic phenomena versus other mechanisms of oxygen deprivation to the myocardium. Due to the focus on the pathophysiological axis, this classification scheme does not directly provide a prognostically meaningful phenotyping of AMI. For example, even if it is generally considered that patients with type 2 MI are more prone to all-cause mortality as compared to type 1 MI, some studies have shown type 1 MI to be associated with poorer outcomes while some other studies found that even non-ischemic myocardial injury may be more fatal than type 1 or type 2 MI. Another meta-analysis indicated that the treatment strategies can differ (such that type 2 MI patients tend to be treated more noninvasively with fewer cardioprotective drugs) resulting in a higher overall mortality in type 2 MI patients. Together, these classification systems tend to over-simplify the wide complexity of presentation and clinical course in AMI patients. Therefore, new and novel ways of AMI phenotyping that are clinically meaningful are urgently required. In that vein, image-based approaches are now being regarded as important tools to aid AMI prognostication.

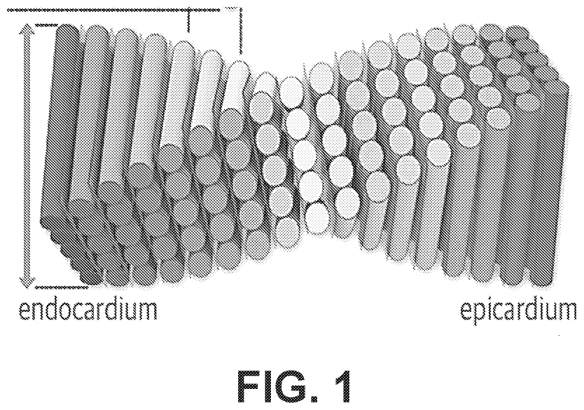

Radiomics based tissue characterization: Cardiac images collated via different modalities might contain information difficult to locate and articulate, which has resulted in substantial effort being spent on cardiac imaging for classification and prognostication. Quantitative features extracted from ultrasound images have been shown to be useful to differentiate normal, malignant and benign tissues. Acoustical, textural and shape features have been evaluated to differentiate malignant melanoma from benign melanocytic tumors. A combination of seven relevant features were identified, yielding an accuracy of 82.4%. Similar accuracies have been observed using quantitative (textural) features to identify malignant thyroid nodules and breast tumors. Notably, a high inter-observer variability in quantitative ultrasound features of the Achilles tendons has been shown. FIG. 1 illustrates an example of texture patterns of ultrasound images and myofiber and matrix architecture. As shown, myocardial fiber and the matrix scaffold can rotate throughout the LV wall. Ultrasound reflections create stronger reflection signal when the direction of propagation is perpendicular to the fiber than parallel. Although the ultrasound backscatter signals may contain useful information regarding myocardial tissue properties, the concentration and arrangement of scatters and backscattered statistics of the ultrasound image have been difficult to define in routine clinical practice.

Radiomics and AMI: Myocardial architecture is altered in patients with MI; however, the experience in the field of radiomics as applied to MI phenotyping is only just emerging. Texture analysis can be used to differentiate between healthy and infarcted myocardium with computed tomography (CT) images, provide objective, accurate and reproducible quantification of images to appropriately risk stratify AMI patients, detect perivascular structural remodeling associated with coronary artery disease and develop imaging biomarkers using artificial intelligence (AI) that can lead to a striking improvement of cardiac risk prediction. In the context of the radiomics-based approaches of cardiac imaging much of the effort to date has been focused on CT and MRI modalities and not much is known about the radiomics information content of cardiac ultrasound. Studies on the radiomics feature extraction from cardiac ultrasound images and their association with clinical endpoints have demonstrated that radiomics-based echocardiographic evaluation holds a substantial promise for AMI prognostication.

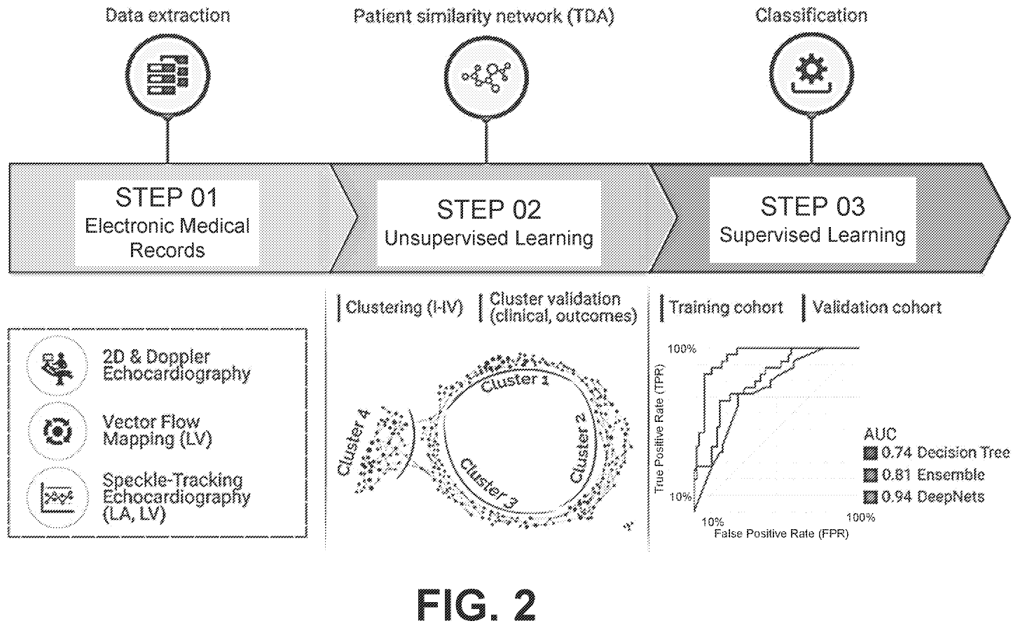

Discovering High Risk Patient Clusters using echocardiography: The use of unsupervised clustering technique can compress the complex dataset derived from echocardiography for creating meaningful representations and identify patient groups with similar presentations and clinical outcomes. Supervised machine learning techniques can be trained for automatically classifying any unknown patients to the distinct patient clusters identified by the patient similarity network. FIG. 2 illustrates an example of a machine-learning (ML) pipeline for patient similarity analysis. Data can be extracted in first step, followed by unsupervised clustering for identifying high risk phenotypes. This can then be followed by development of a supervised classifier for predicting high risk cohorts. The lower panels illustrate a scenario in which 42 echocardiography parameters were fused to develop a similarity network where four clusters (I-IV) were identified with cluster IV showing the highest risk of heart failure and rehospitalization. This can next be followed by development of a classifier model where the Deep NN outperformed the prediction accuracy of other ML classifiers.

A clustering framework for an echocardiographic variable can be used to assess left ventricular diastolic dysfunction in high-risk phenotypic patterns for 866 patients. It was used to identify 2 distinct groups, which conferred agreement with conventional classification. Further cluster analysis further subdivided the cohort in 2 unique cohorts with good agreement with traditional classification. As a result, unique patterns of grouping in diastolic dysfunction was demonstrated. Similarly, patient similarity analysis using topological data analysis (TDA) can be used to identify various phenotypes in aortic stenosis (AS). The TDA algorithm forms a loop, which automatically grouped patients with mild and severe AS on the left and right side. Moderate AS on the top linked both components and bottom sides of this loop with reduced and preserved ejection fraction. These observations were validated in a longitudinal mice data with similar results. Through the utilization of unsupervised learning, it was shown that AS progression is in a state of continuum and is not static in nature.

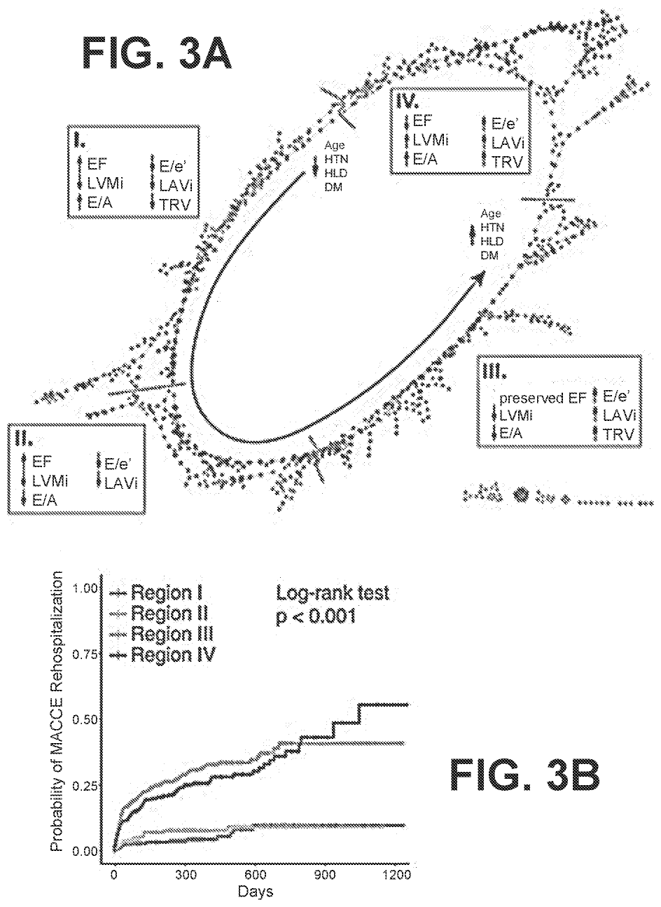

Echocardiography data was collected from 1334 patients to illustrate the potential role of a patient-patient similarity network for mapping cardiac dysfunction without the constraint of any a priori diagnostic system in varying degrees of LV structural and functional remodeling. FIGS. 3A and 3B illustrate an example of unsupervised patient clustering using topological data analysis (TDA) in heart failure patients. Specifically, the TDA model clustered the multiparametric data without utilizing a hierarchical structure or branching tree but rather meaningfully represented the geometry of the data based on the similarity of the patients. Remarkably, the nodes clustered to produce a network in the form of a loop. Moreover, this loop demonstrated the relationships with the outcome of interest, suggesting a valid method of risk stratification for patients.

As shown in FIG. 3A, TDA resamples the disease space multiple times to identify similar patients and link them to nodes (circles). The potential value of this loop for individualized prediction was further illustrated. Using a group of patients with longitudinally collected echocardiographic studies in which the subjects were sampled in different stages of cardiac dysfunction, the analysis suggests that this looped space may also represent the periodic or recurrent behavior of the disease, thereby tracing the path that patients travel through cycles of worsening cardiac function and recovery. The patient-patient network was divided into four distinct regions (I-IV) with varying clinical and echocardiographic characteristics as shown in FIG. 3A. In FIG. 3B, Kaplan-Meier curves of the four regions show varying major adverse cardiac and cerebrovascular event (MACCE) related rehospitalization.

These observations were extended using a similarity network to fuse a total of forty-two echocardiography features including 2-dimensional and Doppler measurements, LV and atrial speckle-tracking and vector flow mapping data obtained in 297 patients (see FIG. 2). This analysis revealed that automated computational methods for phenotyping are an effective strategy to fuse multidimensional parameters of LV structure and function. It can identify distinct cardiac phenogroups in terms of clinical characteristics, cardiac structure and function, hemodynamics, and outcomes.

Applications of Supervised Machine Learning: Consider the range of capabilities of supervised learning. For example, an ensemble algorithm can be employed for distinguishing hypertrophic cardiomyopathy (HCM) from a heart. An associate memory classifier ML algorithm may also be used for differentiating constrictive pericarditis from restrictive cardiomyopathy. An ensemble algorithm can also be utilized for automatic assessment of left ventricular filling pressures from 2-dimensional cardiac ultrasound images. The potential of LV function assessment using strain imaging has been demonstrated to be a robust predictor of complications and recovery beyond the conventional metrics of standard echocardiography. Furthermore, a machine learning technique can be utilized to assess the unique arrangement of pixels (myocardial texture) as high dimensional data to detect unique patient phenotypes. An innovative combination of echocardiographic texture analysis and machine learning was employed to predict structural and functional attributes of cardiac remodeling in 531 patients of whom nearly ⅓ had advanced heart failure. The myocardial feature extraction was successful in 97% cases, 328 features were extracted for a single patient. Cardiac ultrasound can be utilized for tissue characterization similar to cardiac magnetic resonance imaging.

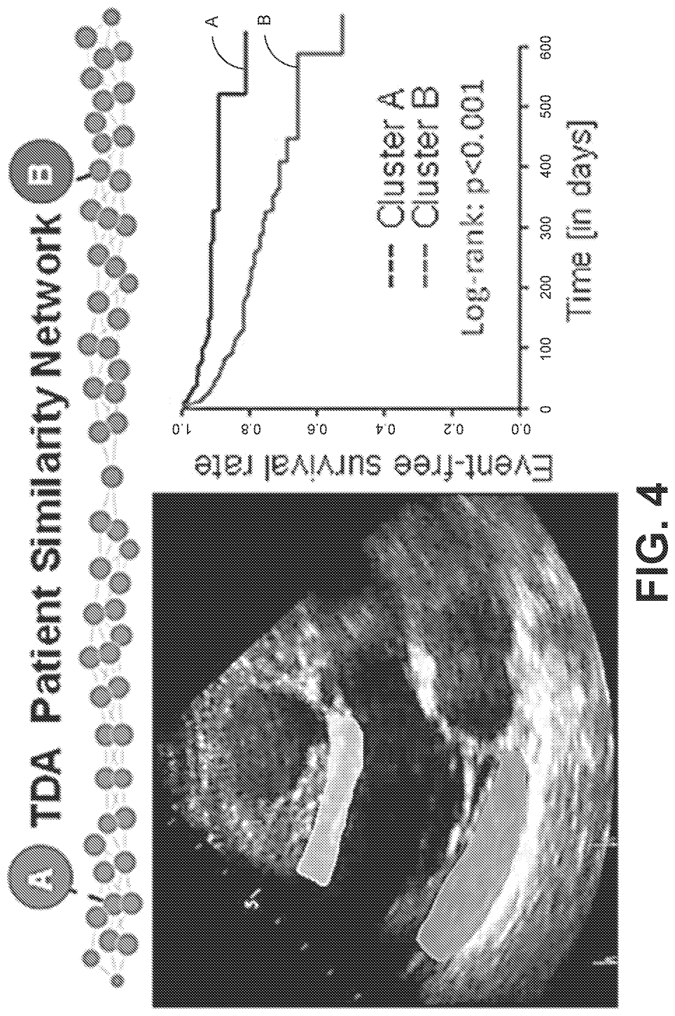

FIG. 4 illustrates an example of a patient similarity network based on myocardial texture features. Kaplan-Meier curve analyses shows that cluster B in a patient similarity network had a significantly higher incidence of the composite of cardiovascular death and major adverse cardiac events compared with cluster A. Texture based features were extracted and integrated using topological data analysis to create a patient similarity network (the shape of a bar) that was geometrically divided into two parts (A & B), which had significantly different clinical and echocardiographic characteristics. Patients with myocardial infarctions were segregated into the high-risk cluster (32.1% vs. 42.9%, P=0.031). During the follow-up period of 301 (ranging from 268 to 323) days, 76 MACEs, including 26 cardiac deaths, were observed. Kaplan-Meier curves showed that MI patients in the high-risk cluster had a significantly higher incidence of cardiac death and MACE than those in the low-risk cluster (P<0.001 by log-rank test, FIG. 4). Although the data collected above included a mixed population of patients (AMI and other cardiomyopathies), these pre-clinical and clinical studies provide preliminary evidence that radiomics features may suitably inform the development of machine-learning applications that could potentially be useful for early risk stratification of AMI patients.

An ensemble of deep learning models was developed to predict high and low-risk heart failure with preserved ejection fraction patient phenogroups using nine echocardiographic parameters in a derivation cohort (n=1,242) and its ability to predict elevated left ventricular (LV) filling pressure validated in a cohort of patients undergoing cardiac catheterization (n=83). In three HFpEF trials, the phenotypic prediction of adverse clinical outcomes (TOPCAT trial, n=518), and the association with cardiac biomarkers and exercise test parameters (NEATHFpEF and RELAX-HF pooled cohort, n=346), was assessed. The model showed robust diagnostic value (Area under the curve:0.997, accuracy:96.0%) for predicting the high and low-risk phenogroups and was better than the 2016 American Society of Echocardiography guidelines for predicting elevated LV filling pressure (c-statistic—0.80 vs. 0.67, P=0.14). In the TOPCAT cohort, the high-risk (vs. low-risk) phenogroup (N=420) had a higher primary composite endpoint on follow-up [Hazard ratio, HR(Confidence interval, CI): 1.92 (1.16-3.22), P=0.01] and associated with a lower risk of the primary composite outcome with Spironolactone therapy [HR(CI): 0.65 (0.46-0.90), P=0.01]. In the pooled RELAX/NEATcohort, the high-risk phenogroup (vs. low-risk) had significantly higher burden of chronic myocardial injury (p<0.001), neurohormonal activation (p<0.001), and lower exercise capacity (p=0.001) independent of other confounders. The integration of conventional echocardiographic indices of LV function, speckle tracking strain and myocardial texture analysis for developing machine learning models can predict adverse features of cardiac remodeling after acute myocardial infarction that are associated with adverse cardiac events.

Other AI-based approaches to AMI phenotyping: Deep learning-based approaches in the context of myocardial infarction include the use of electronic medical records, echocardiography images, coronary calcium, optical coherence tomography have been used to predict myocardial scar tissue, myocardial infarction segmentation and mortality prediction. AI-based approaches to AMI phenotyping has been shown value. For example, it has been shown that deep learning-based risk scores can predict 1-year mortality in AMI patients (both STEMI and NSTEMI) with >90% accuracy [53, 60]. A combination of these approaches with a focus on radiomics-based analysis of echocardiographic images can enhance phenotyping of AMI patients.

The proposed methodology can use existing echocardiographic images by integrating novel texture-based analysis (radiomics) and speckle tracking for phenotyping heterogeneous presentation in AMI and develop unsupervised and supervised machine learning models for AMI risk prediction.

Cardiovascular echo imaging can be converted into clinical aids. For example, the textures of myocardial regions in echo may be analyzed as a potential indicator of diagnosis and prognosis. As described above, the potential of LV function assessment using strain imaging can be a robust predictor of complications and recovery compared to standard echocardiography. The clinical feasibility of cardiac ultrasound fingerprinting was investigated as shown in FIGS. 3A and 3B. A tissue texture-based machine learning framework was used to characterize myocardial functional and structural properties in 531 patients. Myocardial ultrasound feature extraction resulted in 328 features per patient with area under the receiver-operator-characteristics curves (ROC AUC) of 0.83 (sensitivity 91.7% and specificity 72.7%), and 0.87 (sensitivity 88.5% and specificity 71.2%), respectively. These results suggest cardiac ultrasound fingerprinting illustrated in FIG. 4 identifies phenotypic features of LV remodeling from still cardiac ultrasound images.

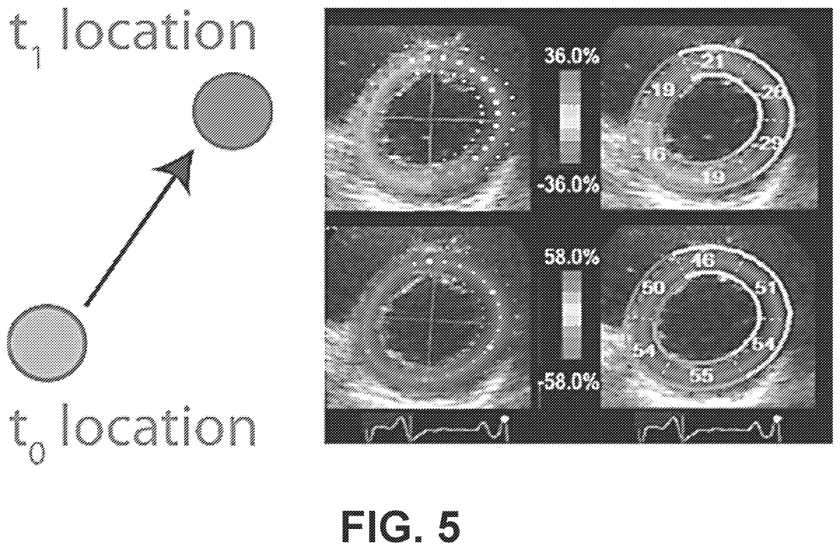

Speckle tracking echocardiography (STE) can use a tracking system based on grayscale B-mode images and can be obtained by automatic measurement of the distance between 2 pixels of an LV segment during the cardiac cycle, independent of the angle of intonation. The underlying principle is that 2-dimensional strain imaging allows rapid and accurate analysis of regional left ventricular (LV) principal strains in the longitudinal, radial, and circumferential directions. FIG. 5 illustrates an example of STE. The rich information content of STE can be converted into prognostically useful information for AMI using AI-based techniques. Thus, integration of conventional echocardiographic indices of LV function, speckle tracking strain and myocardial texture analysis can be used for developing machine learning models that can predict adverse features of cardiac remodeling after acute myocardial infarction that are associated with adverse cardiac events. Machine-learning/deep-learning-based algorithms can be used to extract information from texture analyses of standard echocardiographic images and from STE images for risk-stratification of AMI patients. Repositories of collected echocardiographic studies of AMI patients and equivalent datasets of ECG images and hospitalization medical records can be used to develop and compare accurate ML/DL-based models for risk stratification of AMI patients.

### Analytical Approach Towards Model Development

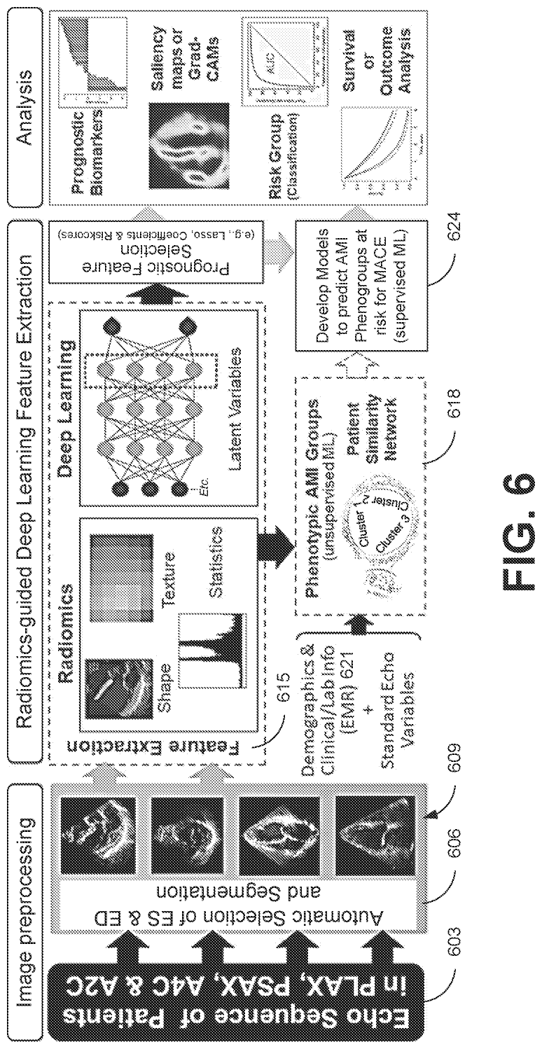

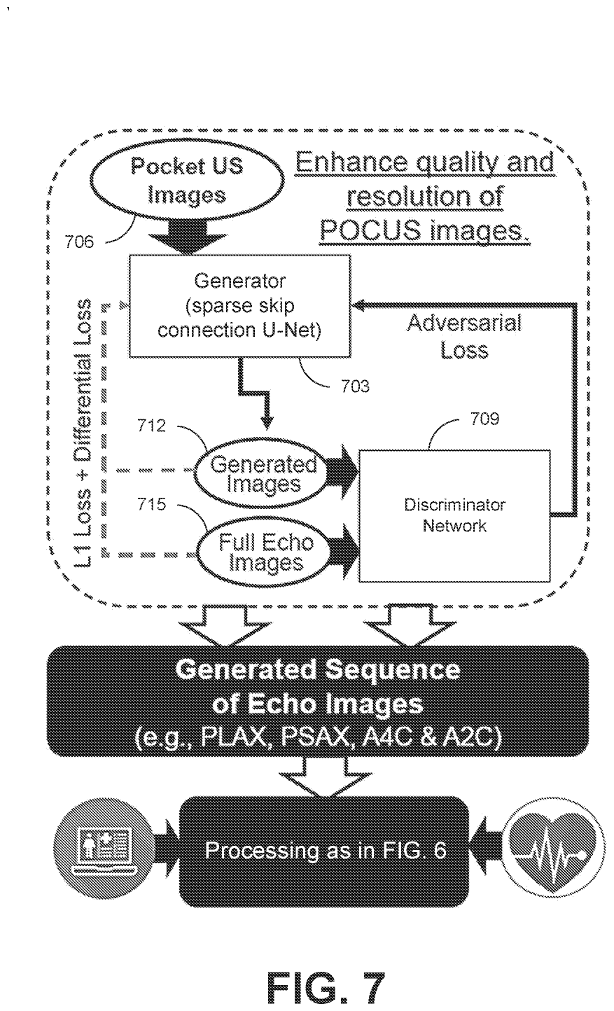

Extraction of Radiomic Features from Echocardiographic Images: FIG. 6 illustrates the schema that can be employed for information extraction from echocardiographic images using the radiomics guided deep neural network pipeline, otherwise known as Cardiac Ultrasound Radiomics Exploration in AMI (CURE-AMI) decision support system. To develop the model, echocardiographic ultrasound patient samples (e.g., about 1000-2000) can be used for performing various tasks detailed. For each patient, four standard two-dimensional videos including the apical four-chamber (A4C) view, the apical two chamber (A2C) view, the parasternal long-axis (PLAX) view and the parasternal short-axis (PSAX) view to evaluate most cardiac functions associated with left ventricular wall segments can be considered at 603. Each video can be composed of two-beat regular rhythm cine-loop including complete diastolic and systolic cycles to evaluate the end-diastolic phase and end-systolic phase, respectively. Non-negative Matrix Factorization (NMF), a dimensionality reduction technique that seeks to find lower parameterizations for high dimensionality data, can be employed at 606 to automatically identify end-systolic (ES, contraction) and end-diastolic (ED, expansion) frames from each of four selected views (PLAX, PSAX, A4C and A2C) of the cardiac ultrasound video. This involves application of Rank-2 and/or robust NMF methods to a series of frames from each view sequence to compute two end-members. The weights and coefficients can then be used to automatically select ES and ED frames at 609 as well as regions of interest (ROIs). Subsequently, the radiomics and STE features from the selected ROI's of the ED/ES frames for the four views for each patient will be extracted at 615 and combined into a dataset, which will form the input for modeling studies.

Predictive Models of AMI Risk Stratification: For unsupervised learning, patient similarity analysis using Topological data analysis (TDA) can be used, a ‘multi-omics’ approach in integrating several conventional and unconventional echocardiographic and/or non-echocardiographic variables. TDA can identify the geometric features and the connectivity among the data points at 618 despite its high variance to demonstrate the progression and thus differs from other clustering techniques that attempt to only break the data into groups without necessarily focusing on the data connectivity. This depiction of a geometric shape with the data continuum and progression in an automated and unsupervised manner can help narrate meaning in the disease space and provide insights. To identify patient similarity network of AMI phenotypic groups, the radiomics and STE features extracted from ultrasound images at are combined with clinical/demographics/traditional echo parameters 621 in order to properly capture the patterns that reflect the diversity of complications that result from AMI.

To simultaneously ingest multiple echocardiographic variables of the dataset and identify phenotypic groups or patient subsets (see, e.g., FIGS. 2-4), TDA can be performed using an automated platform (e.g., Ayasdi Workbench v7.13 and its software development kit, Ayasdi Inc., Menlo Park, CA). In this technique, atop the mathematical underpinning of the notion of shape, unsupervised machine learning can be used at 624 to cluster the patients to generate nodes that are connected via edges if the data points are shared among the nodes. The network can be evaluated for succinct high-resolution description of various outcomes of interest by overlaying gradient of colors on the nodes based on the average measurement values in the nodes. To perform statistical comparisons, the patients can be divided into clusters and then assess the association of these patient clusters with outcomes. The cluster output from TDA can be assigned as a “class label” for developing supervised machine learning model.

Decision tree, ensemble and deepnet model, an optimized deep neural network, can be used for automatic network search and parameter optimization at 624. For this, the data can be randomly divided into training (e.g., 70%) and testing (e.g., 30%) sets. The model can first be trained and tested using conventional echocardiographic and radiomics features only, and subsequently, speckle-tracking and demographic features can be added incrementally. The output variable in the models can be the cluster membership of the patients identified on the patient similarity network. Python, keras and tensorflow frameworks can be used to implement the networks. The models can be targeted to reduce the log-loss function (cross-categorical entropy) and with efforts not to overfit the model to the data. The model providing most accurate classification in the hold-out test set can be retained as the final version of the model. The developed supervised classifier can then be used for predicting any new individual case to identify which patient group or cluster they belong to. These complementary approaches can help identify the patients with high, moderate or low risk of adverse clinical outcomes.

Training data can include patients with in-hospital AMI defined as AMI diagnosis with a hospital stay more than 24 hours after admission to the hospital as identified from collected EMR data. Patients who are aged 50 years or older at the time of the event and admitted to a medical bed service with a diagnosis other than ischemic heart disease by ICD-9 diagnosis codes (410-414) can be included. AMI can be defined as detection of a rise and/or fall of cTn values with at least 1 value above the 99th percentile of upper reference limit with at least 1 of the following: 1) Symptoms of acute myocardial ischemia; 2) New ischemic ECG changes; 3) Development of pathological Q waves; 4) Imaging evidence of new loss of viable myocardium or new regional wall motion abnormality in a pattern consistent with an ischemic etiology; 5) Identification of a coronary thrombus by angiography including intracoronary imaging. Patients discharged to institutionalized care or having a co-existing terminal illness such as cancer can be excluded. Data on previously admitted patients with follow-up information available can be used, including information on MACE events, all-cause mortality and cardiovascular mortality at 30 days, 90 days and 180 days.

Sample size: Machine learning and deep learning tasks do not lend themselves easily to sample size estimations. In general, the more complex the network structure, the better it is to have large sample size. As a rule of thumb, it is a common practice to use an image set of about 1,000 images for deep learning tasks. Much larger ample sizes (e.g., up to about 16,000 images) can be used.

The disclosed methodology can facilitate (1) selection and identification of AMI phenotypic groups based on radiomics and STE echocardiographic features, and (2) use of the knowledge of TDA identified AMI patient clusters to develop models to identify patients with high, moderate or low risk of adverse clinical outcomes. Machine-learning/deep-learning-based algorithms can be used to extract information from deep learning and texture analyses of standard echocardiographic images and from STE images for risk-stratification of AMI patients. Deep learning-based approaches (e.g., convolution neural networks) can be implemented for automated segmentation of the LV wall.

### Development of mHealth Based AMI Prognostication Model

Three benefits support the development of an mHealth-based, deep-learning driven model of AMI risk stratification. First, the convenience and portability of the mHealth devices (e.g., a pocket USG machine) in conducting a quick, bedside evaluation of patient makes for a very attractive alternative to standard echocardiography. Handheld echocardiography devices offer great value in identification and classification of valvular heart diseases, heart structures, and left ventricular functional parameters. It has been determined that there exists a moderate-to-almost perfect correlation between the handheld echocardiography with standard echocardiography with respect to ejection fraction measurement, valve regurgitation identification, left ventricular function and regional wall motion abnormality.

Second, the pocket USG machine is unlikely to possess the full functionality with the same image quality as that obtained through standard echocardiography. However, improving the imaging quality with miniaturized imaging devices can be achieved with machine learning techniques. FIG. 7 illustrates an example for quality and resolution enhancement of POCUS images. Image reconstruction with a generative adversarial network (GAN) model can be used for ultrasound image. The diagnostic/prognostic performance of handheld echocardiography can also be significantly enhanced by adding clinical information of the patient. Third, the use of electronic health records and electrocardiograms can be prognosticators of AMI. The addition of electrocardiographic data can further augment the precision phenotyping by adding complementary imaging related diagnostic and prognostic features. For example, the application of AI with signal-processed electrocardiography (ECG) can aid in predicting myocardial relaxation abnormalities.

The feasibility of signal processed ECG for predicting abnormal myocardial relaxation or left ventricular diastolic dysfunction has been explored. The signal processed ECG provided accurate diagnostic performance in elderly, obese, and hypertensive patients. Machine-learning models were developed that quantitatively estimate myocardial relaxation using clinical and electrocardiography (ECG) variables in the detection of LV diastolic dysfunction. Despite diversity in subjects, the machine-learning model predicted the quantitative values of the LV relaxation velocities (e′) measured by echocardiography in both internal and external test sets (mean absolute error: 1.46 and 1.93 cm/s; adjusted R²=0.57 and 0.46, respectively). The estimated e′ discriminated the guideline-recommended thresholds for abnormal myocardial relaxation and diastolic and systolic dysfunction (LV ejection fraction).

Computed tomography (CT) derived coronary artery calcium (CAC) scoring is a validated measure that correlates well with subclinical coronary atherosclerotic burden. However, the lack of widespread availability and concerns about radiation exposure have limited the universal clinical utilization of CAC. The use of surface ECG indices along with clinical features has been explored to predict clinically used CAC threshold scores (0 and ≥400 Agastan score) in a multi-institutional, prospective patient cohort who underwent surface ECG and CT angiography. The developed ML model was tested to risk stratify a separate cohort of 87 patients referred for invasive coronary angiography. Using an intermediate or higher pretest probability (≥15%) to predict a CAC≥400, the model predicted the presence of significant coronary artery stenosis (P=0.025), the need for revascularization (P<0.001), notably bypass surgery (P=0.021), and major adverse cardiovascular events (P=0.023) during a median follow up period of 2 years. Taken together, features extracted from ECG facilitates vigorous prediction of myocardial relation and this novel strategy can detect patients at risk for left ventricular diastolic dysfunction. Thus, the integration of ML enabled mHealth data (e.g., clinical information, demographics & ECG) and pocket cardiac ultrasound can be expected to increase the diagnostic and prognostic yield of mHealth for medical decision making, predicting relative risk of MACE in AMI patients.

### Prognostic Performance of Machine-Learning Based Model

Prognostic performance of a machine-learning based model combining information from pocket echocardiographic images with electrocardiograph and clinical data derived from electronic health records can be comparable to that provided by a model based on standard echocardiography. While not a substitute to a standard echocardiogram, it would allow for the rational use of a pre-discharge echocardiogram in those patients with high-risk features while the remaining echocardiograms can be done at a subsequent follow-up outpatient visit. As illustrated in FIG. 7, the overall approach can be carried out in two stages: enhancement of images and feature extraction and modeling.

Enhancing the quality of pocket ultrasound images (Stage 1): Compared with the traditional normal-size imaging devices, portable equipment typically produces images with lower spatial resolution, lower contrast, and greater noise. As a result, the poor quality of images has become the major obstacle to the development and further application of portable ultrasound equipment in its prognostic and emergency department. Several techniques including adaptive beamforming, speckle noise reduction and deep learning methods such as CNNs have been introduced to reconstruct images to provide resolution and contrast improvement. Most of these techniques only focus on one or two aspects of image quality. Image reconstruction can use a generative adversarial network (GAN) model to break through the imaging quality limitation of portable devices.

As shown in FIG. 7, GAN models can be used to perform ultrasound image reconstruction to improve the imaging quality of portable ultrasound images. To do this, two GAN generator models are combined, an encoder-decoder model and a U-Net model, to build a sparse skip connection U-Net (SSC U-Net) 703 to improve the quality of images 706 from handheld ultrasound equipment in terms of spatial resolution, contrast, and noise reduction. A discriminator network 709 can provide the generator 703 with adversarial loss based upon the generated images 712 and full echo images 715.

Feature extraction and model development (Stage 2): This stage can follow a similar workflow detailed in FIG. 6. First, ED/ES frames are identified, and ROIs selected from the enhanced pocket US echo sequences. Following this radiomic and STE features from corresponding regions on the ED/ES frames from the four different views can be extracted. Next, the patient similarity network/clusters can be identified using unsupervised learning. TDA based on demographics, clinical information, echocardiographic features and those from the ROI's (i.e., radiomics and STE) extracted by automation can be used for selecting and identifying phenotypic groups. Following this, the echo/radiomics/STE features can be concatenated along with their cluster assignments with the data from electronic clinical records and signal processed data from electrocardiographs. The concatenated data can become input for supervised learning. For supervised learning, Python, keras and tensorflow frameworks can be used to implement the networks. The model will thus depend on the three sources of data (as shown in FIGS. 6 and 7). The model can optimize the log-loss function (cross-categorical entropy) so as not to overfit the model to the data. The model with most accurate classification in the hold-out test set will be retained for predicting new individual cases.

Training data can include patients as those discussed above. A consecutive sampling of patients admitted to a Coronary Care Unit (CCU) or cardiac wards can be adopted. Inclusion criteria can include: (1) age above 21 and below 85; (2) clinical diagnosed and documented AMI as defined above; (3) undergone PCI for the index event; (4) able to provide informed consent. Patients discharge to institutionalized care; co-existing terminal illness such as cancer; and psychiatric or cognitive disorders can be excluded. Follow-up information from the patients can be used, including information on MACE, all-cause mortality and cardiovascular mortality at 30 days, 90 days and 180 days after discharge.

### mHealth Integrated Strategy

The mHealth integrated strategy may be directly compared with standard echocardiography strategy in AMI patients to predict clinically meaningful endpoints. AMIs put enormous time and cost pressures on the hospitals in the United States. Accompanying this fact is the concern for overuse of echocardiography. These two preconditions have generally constrained the use of echocardiography in hospitalized AMI patients such that the standard-of-care is to request a follow-up visit after 7 days to perform echocardiography evaluation. This approach can not only lead to attrition but the lack of echocardiography evaluation at the time of discharge can also fail to inform the clinicians of lurking potential high-risk cardiac phenotypic features at the time of discharge that may be associated with an adverse follow-up events.

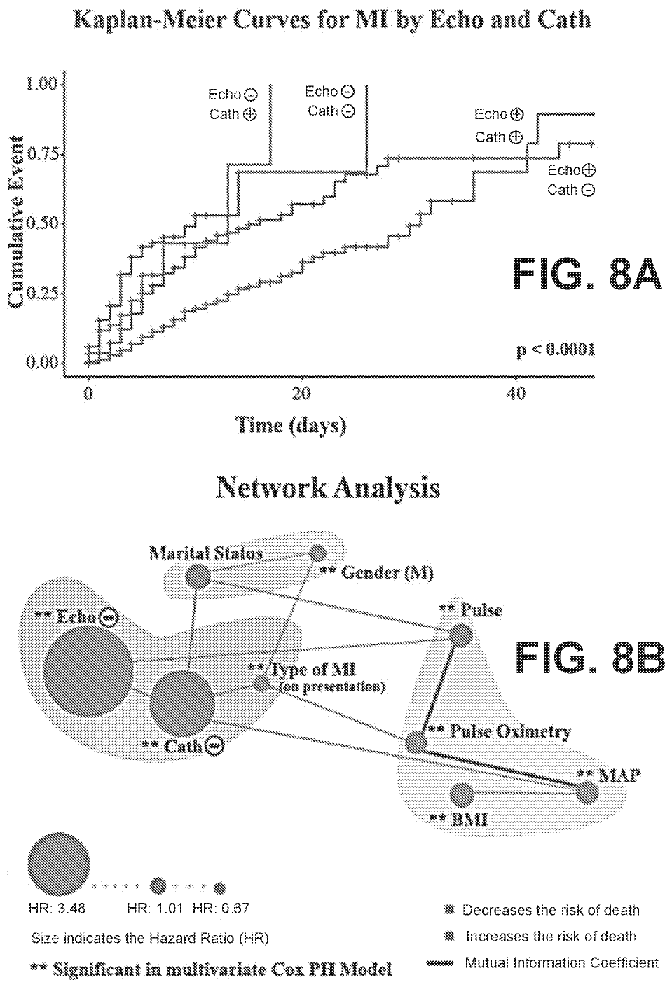

In a large, nationwide, decade-long investigation using the Nationwide Inpatient Sample (NIS) database, it was observed that, in contrast to the expectation, there has been an underuse of echocardiography in US hospitalizations. FIGS. 8A and 8B illustrate the implications of the underuse of echocardiography. FIG. 8A shows Kaplan-Meier curves for MI and FIG. 8B illustrates network analysis. An mHealth strategy can be used where the performance of pocket USG is enriched with AI-enabled high-risk features extracted from ECG (including signal-processed surface 12-lead ECG) and high-risk clinical features extracted from the electronic medical record associated with hospitalization can help identify high-risk patient phenotypic features even in the absence of a standard echocardiographic evaluation. The use of AI-enabled mHealth evaluation (including ECG and pocket ultrasound) at the time of discharge can be comparable to the standard-of-care full echocardiographic evaluation during hospitalization in terms of feasibility, accuracy of risk stratification, acceptability amongst medical professionals without incurring added hospitalization costs.

### Statistical Analysis Plan

Analyses related to agreement between standard echo-based and pocket USG-based risk-stratification: Both the standard-echo-based model and the pocket-USGE based model can yield risk-stratification in a tri-state classification (high-risk, moderate-risk, low-risk). A weighted Cohen's kappa can be used to estimate the extent of agreement between these two methods. These models can also yield the estimated probability of the three outcomes in each patient. These estimated probabilities can be used and converted into log-odds (to ensure normal distribution) for use with Pearson's correlation coefficient and Bland-Altman plots to examine for any residual biases between the two models. Together, these analyses will help estimate the degree of concordance between the two models on the same set of patients.

Analyses related to association of predicted risk profiles with clinical outcomes: The clinical use of the AI-based methods can be used to predict adverse outcomes within 6 months of follow-up. Three events (MACE, all-cause mortality and cardiovascular mortality) can be assessed at three time-points (30, 90 and 180 days of discharge) and each of these nine outcomes (3 events×3 time points) can be used in the association analyses. Associations may be tested in a logistic regression framework (unconditional as well as multinomial to predict the combinations of events). These analyses can demonstrate the noninferiority of pocket-USG based risk-stratification as compared to the standard echo-based risk stratification. These analyses can be conducted both in the “comparative component” and separately in the “predictive component” for the pocket USG based risk-stratification.

Analyses related to physician's acceptability of pocket USG use at discharge: The data related to acceptability can be collected and analyzed semi-quantitively using percentages. The AI-based risk profiles can also be associated with physician's acceptability scores to investigate if specific patient subsets exist where physician's acceptability of the pocket-USG based classifier needs improvement.

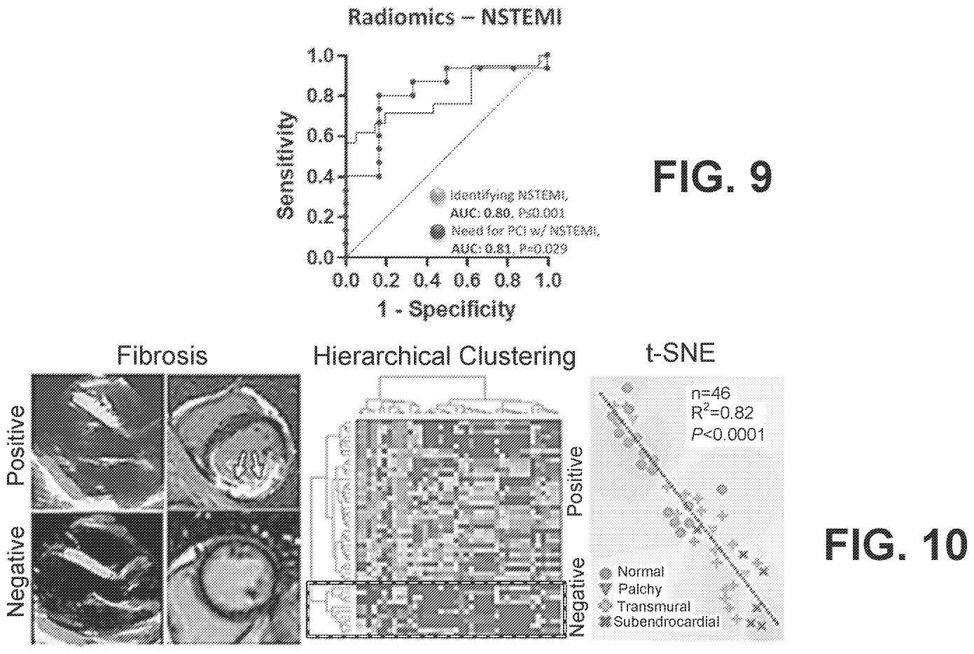

Preliminary Data on POCUS-based Radiomics and Automated AI: Recently, using a translational approach, the quantitative assessments of myocardial ultrasonic morphometry and the textural features in cardiac ultrasound images were explored to predict LV dysfunction by implementing an automated machine learning (ML) pipeline as a first step to aid the high-throughput screening of POCUS images (n=943). The subsequent exploration of this concept in clinical images obtained using different POCUS devices (n=275) and high-end ultrasound systems (n=484) suggested that the ultrasonic texture-based changes may be particularly robust to variation in image acquisition settings and quality. These data suggest that POCUS may be a good target for deep-learning derived refinement for radiomics signature extraction. In a preliminary dataset, the POCUS-derived model was able to differentiate patients presenting without ST-elevation changes on ECG (NSTEMI) (n=21) from a control, matched cohort (n=21) (AUC: 0.80, P≤0.001), as shown in FIG. 9.

Preliminary Data on Echo-derived Radiomics and CMR for tissue characterization: In a previous study, 89 retrospectively identified patients who had undergone CMR and echocardiography within 48 hours were used as the training set and 40 independent prospective patients were used as the test set. In the test set, the developed machine-learning model (an ensemble of 2 LASSO regressions) predicted late gadolinium enhancements with a ROC AUC of 0.84 (sensitivity 86.4%, and specificity 83.3%). More specifically, in 46 patients with MI, hierarchical clustering of echo-extracted radiomics features separated patients with LGE. It delineated the severity of infarcted myocardium within the myocardial segment. FIG. 10 illustrates hierarchical clustering of the echo-derived radiomic features showing the ability of texture patterns in discriminating the presence or absence of LGE (highlighted in box) on CMR scans and t-SNE plot visualizing the clusters of scar tissue depending on their transmural distribution in AMI patients. This further supports the use of radiomics features in identifying and delineating the extent of the infarcted region as identified using CMR (e.g., myocardial scar: Yes/No and the location of the scar tissue).

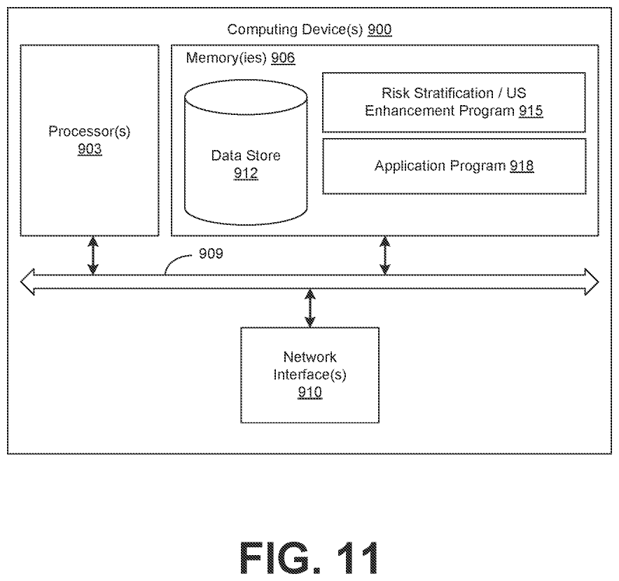

With reference to FIG. 11, shown is a schematic block diagram of a computing device 900 that can be utilized to enhance pocket US and evaluate risk stratification using the described techniques. In some embodiments, among others, the computing device 900 may represent a mobile device (e.g. a smartphone, tablet, computer, etc.). Each computing device 900 includes at least one processor circuit, for example, having a processor 903 and a memory 906, both of which are coupled to a local interface 909. To this end, each computing device 900 may comprise, for example, at least one server computer or like device. The local interface 909 may comprise, for example, a data bus with an accompanying address/control bus or other bus structure as can be appreciated.

In some embodiments, the computing device 900 can include one or more network interfaces 910. The network interface 910 may comprise, for example, a wireless transmitter, a wireless transceiver, and a wireless receiver. As discussed above, the network interface 910 can communicate to a remote computing device using a Bluetooth protocol. As one skilled in the art can appreciate, other wireless protocols may be used in the various embodiments of the present disclosure.

Stored in the memory 906 are both data and several components that are executable by the processor 903. In particular, stored in the memory 906 and executable by the processor 903 are a risk stratification/US enhancement program 915, application program 918, and potentially other applications. Also stored in the memory 906 may be a data store 912 and other data. In addition, an operating system may be stored in the memory 906 and executable by the processor 903.

It is understood that there may be other applications that are stored in the memory 906 and are executable by the processor 903 as can be appreciated. Where any component discussed herein is implemented in the form of software, any one of a number of programming languages may be employed such as, for example, C, C++, C #, Objective C, Java®, JavaScript®, Perl, PHP, Visual Basic®, Python®, Ruby, Flash®, or other programming languages.

A number of software components are stored in the memory 906 and are executable by the processor 903. In this respect, the term “executable” means a program file that is in a form that can ultimately be run by the processor 903. Examples of executable programs may be, for example, a compiled program that can be translated into machine code in a format that can be loaded into a random access portion of the memory 906 and run by the processor 903, source code that may be expressed in proper format such as object code that is capable of being loaded into a random access portion of the memory 906 and executed by the processor 903, or source code that may be interpreted by another executable program to generate instructions in a random access portion of the memory 906 to be executed by the processor 903, etc. An executable program may be stored in any portion or component of the memory 906 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 906 is defined herein as including both volatile and nonvolatile memory and data storage components. Volatile components are those that do not retain data values upon loss of power. Nonvolatile components are those that retain data upon a loss of power. Thus, the memory 906 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 903 may represent multiple processors 903 and/or multiple processor cores and the memory 906 may represent multiple memories 906 that operate in parallel processing circuits, respectively. In such a case, the local interface 909 may be an appropriate network that facilitates communication between any two of the multiple processors 903, between any processor 903 and any of the memories 906, or between any two of the memories 906, etc. The local interface 909 may comprise additional systems designed to coordinate this communication, including, for example, performing load balancing. The processor 903 may be of electrical or of some other available construction.

Although the risk stratification/US enhancement program 915 and the application program 918, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

Also, any logic or application described herein, including the risk stratification/US enhancement program 915 and the application program 918, that comprises software or code can be embodied in any non-transitory computer-readable medium for use by or in connection with an instruction execution system such as, for example, a processor 903 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the risk stratification/US enhancement program 915 and the application program 918, may be implemented and structured in a variety of ways. For example, one or more applications described may be implemented as modules or components of a single application. For example, separate applications can be executed for the PH and Radon transform workflows as illustrated in FIGS. 6 and 7. Further, one or more applications described herein may be executed in shared or separate computing devices or a combination thereof. For example, a plurality of the applications described herein may execute in the same computing device 900, or in multiple computing devices in the same computing environment. Additionally, it is understood that terms such as “application,” “service,” “system,” “engine,” “module,” and so on may be interchangeable and are not intended to be limiting.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

1. A system, comprising:
a handheld echocardiography device configured to generate ultrasound (US) images of a patient; and
processing circuitry comprising a processor and memory, the processing circuitry configured to:
receive the US images from the handheld echocardiography device;
generate enhanced echo images from the US images using a generative adversarial network (GAN) model; and
determine a major adverse cardiac event (MACE) risk for the patient based upon the enhanced echo images.

2. The system of claim 1, wherein the GAN model comprises a sparse skip connection U-Net model.

3. The system of claim 2, wherein the sparse skip connection U-Net model combines an encoder-decoder model and a U-Net model.

4. The system of any of claims 1-3, wherein the enhanced echo images comprise an apical four-chamber (A4C) view, an apical two chamber (A2C) view, a parasternal long-axis (PLAX) view and a parasternal short-axis (PSAX) view.

5. The system of any of claims 1-4, wherein the MACE risk determination comprises:
extracting features from the enhanced echo images;
analyzing phenotypes based at least in part upon the extracted features; and
predicting risk of the MACE using a machine learning model.

6. The system of claim 5, wherein the phenotype analysis comprises patient similarity analysis using topological data analysis (TDA).

7. The system of claim 6, wherein the predicted risk is based upon the patient similarity analysis and clinical information associated with the patient.

8. The system of any of claims 5 and 6, wherein extracting features of the enhanced echo images comprises:
identifying end-systolic (ES) and end-diastolic (ED) frames from the enhanced echo images and selecting regions of interest (ROIs) from the identified ED/ES frames; and
performing texture-based analysis (radiomics) and speckle tracking for phenotyping heterogeneous presentation.

9. The system of claim 8, wherein the ES and ED frames are identified using non-negative matrix factorization.

10. A method, comprising:
receiving ultrasound (US) images of a patient obtained with a handheld echocardiography device;
generating enhanced echo images from the US images using a generative adversarial network (GAN) model; and
determining a major adverse cardiac event (MACE) risk for the patient based upon the enhanced echo images.

11. The method of claim 10, wherein the US images are received from the handheld echocardiography device.

12. The method of any of claims 10 and 11, wherein the GAN model comprises a sparse skip connection U-Net model.

13. The method of claim 12, wherein the sparse skip connection U-Net model combines an encoder-decoder model and a U-Net model.

14. The method of any of claims 10-13, wherein the enhanced echo images comprise an apical four-chamber (A4C) view, an apical two chamber (A2C) view, a parasternal long-axis (PLAX) view and a parasternal short-axis (PSAX) view.

15. The method of any of claims 10-14, wherein the MACE risk determination comprises:
extracting features from the enhanced echo images;
analyzing phenotypes based at least in part upon the extracted features; and
predicting risk of the MACE using a machine learning model.

16. The method of claim 15, wherein the phenotype analysis comprises patient similarity analysis using topological data analysis (TDA).

17. The method of claim 16, wherein the predicted risk is based upon the patient similarity analysis and clinical information associated with the patient.

18. The method of any of claims 15 and 16, wherein extracting features of the enhanced echo images comprises:
identifying end-systolic (ES) and end-diastolic (ED) frames from the enhanced echo images and selecting regions of interest (ROIs) from the identified ED/ES frames; and
performing texture-based analysis (radiomics) and speckle tracking for phenotyping heterogeneous presentation.

19. The method of claim 18, wherein the ES and ED frames are identified using non-negative matrix factorization.

20. The system of claim 18, wherein the features extracted from corresponding ROIs of the identified ED/ES frames comprise left ventricular (LV) geometry.
